(19) United States
(12) Patent Application Publication
(10) Pub. No.: US 2022/0128158 A1
(43) Pub. Date: Apr. 28, 2022

(54) Pinching Device for Flexible Tubing

(71) Applicant: BioFlex Solutions, LLC, Hackettstown, NJ (US)

(72) Inventors: Jason C. Nisler, Newton, NJ (US); Ralph M. Meola, Hackettstown, NJ (US); Ian Colquhon, Frelinghuysen, NJ (US)

(73) Assignee: BioFlex Solutions, LLC, Hackettstown, NJ (US)

(21) Appl. No.: 17/507,203

(22) Filed: Oct. 21, 2021

Related U.S. Application Data
(60) Provisional application No. 63/105,229, filed on Oct. 24, 2020.

(51) Int. Cl. F16K 7/06

(52) U.S. Cl. CPC F16K 7/063

(57) ABSTRACT

The pinch clamp has a ratchet mechanism for selectively securing two jaw assemblies of the pinch clamp together. The ratchet mechanism includes a ratchet with a plurality of notches fixedly mounted on an end of one jaw assembly and a pawl member integrally mounted on an end of the other jaw assembly for selectively engaging in one of the notches of the ratchet. A finger release portion extends integrally from the pawl member for moving the pawl member from engagement with the ratchet.

This is a Non-Provisional Patent Application and claims the benefit of Provisional Patent Application 63/105,229, filed Oct. 24, 2020.

This invention relates to a pinching device and more particularly to a pinching device for temporarily closing flexible tubing.

### BACKGROUND OF THE INVENTION

Pinching devices for flexible tubing are well known for use in the food and pharmaceutical industry for the temporary closure of fluid lines. Typically, pinching devices are used for specific sizes of tubing wherein most require the tubing to be inserted prior to connection.

U.S. Pat. No. 9,879,813 describes a pinching device that does not require the insertion of tubing through the device and that may be placed at various locations along fluid lines while actively in use. The pinching device is constructed with two members that pivot relative to each other to close and open on a flexible tubing with a locking nut assembly to lock the members in place.

### OBJECTS OF THE INVENTION

It is an object of the invention to provide an improved pinching device for flexible tubing.

It is another object of the invention to simplify the locking together of a pair of pivotal members of a pinching device.

It is another object of the invention to simplify the manufacture of a pinching device for flexible tubing.

### BRIEF SUMMARY OF THE INVENTION

Briefly, the invention provides a pinching device for flexible tubing that comprises a pair of jaw assemblies that are pivotally connected to each other to close on a flexible tubing and a ratchet mechanism for securing the jaw assemblies in place.

A first jaw assembly has a first pinching bar and a pivot at one end of said pinching bar and the second jaw assembly has a second pinching bar opposite the pinching bar of the first jaw assembly for pinching a flexible tubing therebetween. In addition, the second jaw assembly has a hook portion at one end of the second pinching bar that is received on the pivot of the first jaw assembly for pivoting of the jaw assemblies relative to each other between an open position and a closed position with a flexible tubing therebetween.

The ratchet mechanism includes a ratchet with a plurality of notches fixedly mounted on an end of the first jaw assembly opposite the pivot and a pawl member integrally mounted on an end of the second jaw assembly opposite the hook portion for selectively engaging in one of the notches of the ratchet. Engagement of the pawl member in a selected notch effects a locking of the two jaw assemblies together in the closed position.

The second jaw assembly also includes a finger release portion that extends integrally from the pawl member for recovering the pawl member manually from engagement with the ratchet.

In order to incorporate the ratchet mechanism in the pinching device, the first jaw member has a bifurcated end opposite the pivot for receiving the ratchet. In addition, a hollow pin passes into one side of the bifurcated end and into the ratchet to mount the ratchet in the bifurcated end. The ratchet is sized and shaped to be held in a stet nary manner within the bifurcation once the hollow pin is passed into the ratchet.

In addition, a rivet passes into an opposite side of the bifurcated end of the first jaw assembly and into the hollow pin to secure the hollow pin in the first jaw assembly.

The pawl member is formed as an integral extension of the pinching bar of the second jaw assembly with a leg extending in depending manner from the pinching bar and a detent extending from the leg towards the ratchet. In addition, the finger release portion extends integrally from the leg opposite the detent for moving the detent from engagement with the ratchet.

These and other objects and advantages of the invention will become more apparent from the following detailed description taken in conjunction with the accompanying drawings wherein:

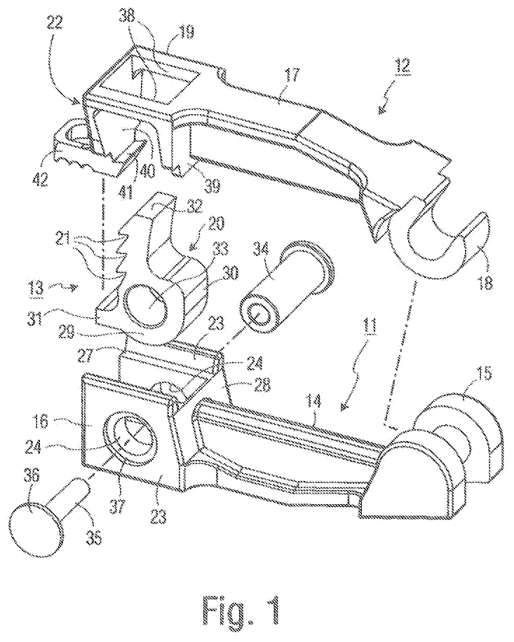

FIG. 1 illustrates an exploded view of a pinching device in accordance with the invention;

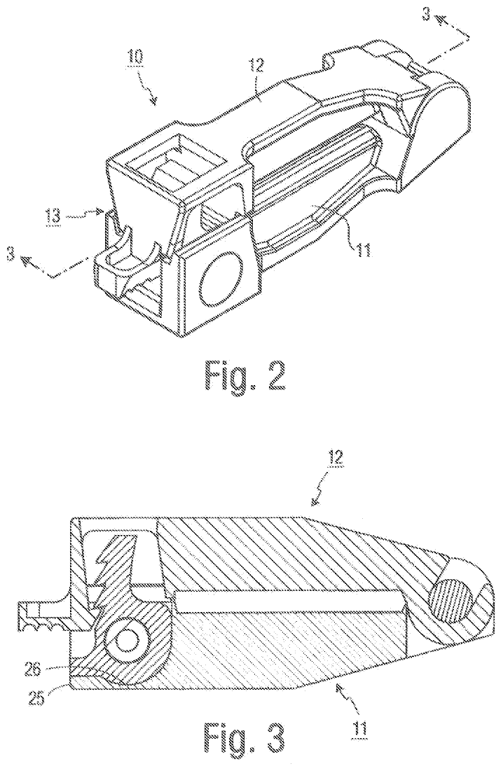

FIG. 2 illustrates a perspective view of the pinching device of FIG. 1; and

FIG. 3 illustrates a cross-sectional view of the pinching device of FIG. 2 in a closed condition.

Referring to FIGS. 1 and 2, the pinching device 10 comprises two jaw assemblies 11, 12 and a ratchet mechanism 13.

The pair of jaw assemblies 11, 12 are pivotally connected to each other to close on a flexible tubing (not shown) and the ratchet mechanism 13 serves to secure the jaw assemblies 11, 12 together in place while pinching the flexible tubing (not shown). Typically, the jaw assemblies 11, 12 are made of a plastic material and are constructed to function in a manner as described ire U.S. Pat. No. 9,879,813.

Referring to FIG. 1, the lower jaw assembly 11, as viewed, has a pinching bar 14 with a pivot 15 at one end and a bifurcation 16 at an opposite end.

The second or upper jaw assembly 12, as viewed, has a pinching bar 17 opposite the pinching bar 14 of the lower jaw assembly 11 for pinching a flexible tubing (not shown) therebetween, a hook portion 18 at one end of the pinching bar 16 for reception on the pivot 15 of the lower aw assembly 11 and a bifurcation 19 at an opposite end of the pinching bar 17 for mating with the bifurcation 16 of the lower jaw assembly 11.

The ratchet mechanism 13 includes a ratchet 20 with a plurality of notches fixedly mounted on an end of the lower jaw assembly 11 opposite the pivot 15 and a pawl member 22 integrally mounted on an end of the upper jaw assembly 12 opposite the hook portion 17 for selectively engaging in one of the notches 21 of the ratchet 20.

The ratchet 20 is a one piece body made of a material suitable for use in the food and pharmaceutical industry for the temporary closure of fluid lines. As illustrated in FIG. 1, the ratchet 20 is sized and shaped t be mounted in the bifurcation 16 of the lower jaw assembly 11 in a stationary upright manner.

The bifurcation 16 at the end of the lower jaw assembly 11 is formed of two parallel side walls 23, each of which has an aperture 24, and a floor 25 that extends between the side wails 23 with an intermediately disposed curvilinear surface 26 (see FIG. 3). Each side wall 23 has an internally disposed ledge 27 at an upper end, as viewed, extending the length of the side wall 23. In addition, a back wall 28 extends between the side walls 23 to form a box-like recess with an open side.

The ratchet 20 has a curvilinear base 29 of a shape to be matingly received on the surface 26 of the floor 23, a flattened back 30 of a shape to lie against the back wall 28, a leg 31 that projects forwardly from the curvilinear base 29 to be received on the floor 25 and an upstanding neck 32 that contains the notches 21 on one side.

Alternatively, the ratchet may be made integrally with the bifurcation 16 at the end of the pinching bar 14 to reduce he number of parts for production assembly.

As illustrated in FIG. 1 each of the notches 21 is under-cut to form a downwardly directed upper flank, as viewed.

In addition, the ratchet 20 has a bore 33 extending therethrough above the base 29 and below the neck 32.

In order to secure the ratchet, 20 in the lower jaw assembly 11, a hollow cylindrical pin 34 is passed through an aperture 24 in a side wall 23 of the bifurcation 16 of the lower jaw assembly 11 and into the bore 33 of the ratchet 20. In addition, a rivet 35 is passed through the aperture 24 in the opposite side wall 23 and into the hollow pin 34 to secure the hollow pin 34 in the lower jaw assembly 11. As illustrated, the rivet 35 has a head 36 for seating in an annular recess 37 about the mouth of the aperture 24 in the side wall 23.

The bifurcation 19 at the end of the upper jaw assembly 12 is formed of a pair of parallel arms 38 that extend from a transverse wall 39 at the end of the pinching bar 16.

The pawl member 22 has a leg 40 that extends in depending manner from the ends of the arms 38 of the bifurcation 19. As shown, the leg 40 is of plate shape and is disposed in spaced parallel manner to the transverse well 39 at the end of the pinching bar 16. The shape and thickness of the leg 40 is sufficient to allow the leg 40 to flex outwardly of the end of the pinching bar 16 under a manually applied force.

The pawl member 22 also has a detent 41 that extends from the leg 40 towards the transverse wall 39 and the ratchet 20. The detent 41 extends across the bottom edge of the leg 40 and is shaped to fit into a selected notch 21 of the ratchet 20 under the downwardly directed upper flank of the notch.

In use, when the two jaw assemblies 11, 12 are brought into closed condition on a tubing (not shown) the leg 40 of the pawl member 22 flexes as the detent 41 rides down the row of notches 21 until stopping in a notch 21 corresponding to the closed position of the jaw assemblies 11, 12 on the tubing. Due to the undercut of the notch 21, the detent 41 is biased by the pinched tubing into a clamped engagement with the upper flank of the notch 21 and held against inadvertent release.

In order to release the ratchet 20 from the pawl member 22, a finger release portion 42 extends integrally from the leg 40 opposite the detent 41 for moving the defiant 41 out of engagement with the ratchet 13. In this respect, manually lifting the finger release portion 42 causes the detent 41 to move out of an engaged notch 21 to allow the upper jaw assembly 12 to be pivoted away from the lower jaw assembly 11 to the open position to permit release from a pinched tubing.

The pinching device 10 may be assembled from parts that can be simply manufactured. For example, the two jaw assemblies 11, 12 and ratchet 20 may be molded of a suitable plastic material. The pin 34 and rivet 35 may be made of plastic or a suitable metal.

Assembly of the pinching device 10 requires mounting of the upper jaw assembly 12 on the lower jaw assembly 11, insertion of the ratchet 20 in the bifurcated end of the lower jaw assembly 11 and pinning of the ratchet 20 in place by the pin 34 and rivet 35. These steps may also be reversed with the ratchet 20 being mounted first in the lower is jaw assembly 11.

The invention thus provides an improved pinching device for flexible tubing that simplifies the locking together of a pair of jaw assemblies of a pinching device.

Further, the invention provides an improved pinching device for flexible tubing that may be manufactured in a simplified manner.

What is claimed is:

1. A pinching device for a flexible tubing comprising
a first jaw assembly having a first pinching bar and a pivot at one end of said pinching bar;
a second jaw assembly having a second pinching bar opposite said first pinching bar for pinching a flexible tubing therebetween and a hook portion at one end of said second pinching bar received on said pivot of said first jaw assembly, and
a ratchet mechanism for selectively securing said second jaw assembly to said first jaw assembly, said ratchet mechanism including a ratchet with a plurality of notches fixedly mounted on an end of said first jaw assembly opposite said pivot and a pawl member integrally mounted on an end of said second jaw assembly opposite said hook portion for selectively engaging in one of said notches of said ratchet.

2. A pinching device as set forth in claim 1 wherein said ratchet mechanism further includes a finger release portion extending integrally from said pawl member for moving said pawl member from engagement with said ratchet.

3. A pinching device as set forth in claim 1 wherein said first jaw member has a bifurcated end opposite said pivot receiving said ratchet therein.

4. A pinching device as set forth in claim 3 further comprising a hollow pin passing into one side of said bifurcated end and into said ratchet to mount said ratchet in said bifurcated end and a rivet passing into an opposite side of said bifurcated end and into said hollow pin to secure said hollow pin in said first jaw assembly.

5. A pinching device for a flexible tubing comprising
a first jaw assembly of plastic material having a first pinching bar and a pivot at one end of said pinching bar;
a second jaw assembly of plastic material having a second pinching bar opposite said first pinching bar for pinching a flexible tubing therebetween and a hook portion at one end of said second pinching bar received on said pivot of said first jaw assembly for pivoting of said first jaw assembly and said second jaw assembly relative to each other between an open position and a closed position with a flexible tubing therebetween; and
a ratchet mechanism for selectively securing said second jaw assembly to said first jaw assembly, said ratchet mechanism including a ratchet with a plurality of notches fixedly mounted on an end of said first jaw assembly opposite said pivot and a pawl member integrally mounted on an end of said second jaws assembly opposite said hook portion for selectively engaging in one of said notches of said ratchet.

6. A pinching device as set forth in claim 5 wherein said pawl member leas a leg extending in depending manner from said second pinching bar and a detent extending from said leg towards said ratchet.

7. A pinching device as set forth in claim 6 wherein said ratchet mechanism further includes a finger release portion extending integrally from said leg opposite said detent for moving said detent from engagement with said ratchet.
